(19) United States
(12) Patent Application Publication
(10) Pub. No.: US 2019/0321417 A1
(43) Pub. Date: Oct. 24, 2019

(54) BACTERIAL PROBIOTIC TO DETOXIFY HERBICIDES AND INSECTICIDES

(71) Applicant: PRESIDENT AND FELLOWS OF HARVARD COLLEGE, Cambridge, MA (US)

(72) Inventors: Robert M. Brucker, Melrose, MA (US); Guan-Hong Wang, Cambridge, MA (US)

(73) Assignee: PRESIDENT AND FELLOWS OF HARVARD COLLEGE, Cambridge, MA (US)

(21) Appl. No.: 16/388,001

(22) Filed: Apr. 18, 2019

Related U.S. Application Data
(60) Provisional application No. 62/659,374, filed on Apr. 18, 2018.

(51) Int. Cl. A61K 35/741; A61K 9/00; A01N 25/32; A01N 63/00; A61P 39/02

(52) U.S. Cl. CPC A61K 35/741; A61K 9/0073; A61K 9/0053; A61K 2035/115; A01N 63/00; A61P 39/02; A01N 25/32

(57) ABSTRACT

Described herein are methods and compositions for the use of detoxifying an herbicide and/or promoting resistance to an herbicide. Aspects of the invention relate to administering to a subject in need thereof a composition comprising viable S. marcescens bacteria and/or P. protegens bacteria.

### CROSS-REFERENCE TO RELATED APPLICATIONS

This application claims benefit under 35 U.S.C. § 119(e) of U.S. Provisional Application No. 62/659,374 filed Apr. 18, 2018, the contents of which are incorporated herein by reference in their entirety.

### SEQUENCE LISTING

The instant application contains a Sequence Listing which has been submitted in ASCII format via EFS-Web and is hereby incorporated by reference in its entirety. Said ASCII copy, created on Apr. 18, 2019, is named 002806-091770USPT_SEQ_listing_ST25.txt and is 3,951 bytes in size.

### FIELD OF THE INVENTION

The field of the invention relates to the use of probiotics to modify the gut microflora.

### BACKGROUND

Pollinators are crucial for the maintenance of wild plants and agricultural productivity; 80% of wild plants and up to 75% of main crops consumed by humans are dependent upon pollination. However, the global pollinator population is drastically declining and can be felt throughout the globe. A recent study from Germany surveyed 63 sites over 27 years and found an average 76% decline in airborne insect biomass, and just within the US there has been a 59% loss of domestic honeybee colonies between 1947-2005.

While the cause of pollinator decline remains under investigation, pesticide exposure and infectious diseases are implicated as co-factors. Pesticides are widely used throughout the US and worldwide. For example, Atrazine is the second most sold (per tonnage) pesticide globally and is primarily used for corn production and orchards in the U.S. The U.S. Environmental Protection Agency has deemed the continuous daily average of 3 ppb, parts per billion, in fresh and potable water acceptable, and has detected the herbicide in over 78% of drinking water throughout the Midwestern United States. The impact of Atrazine on animals and humans is unclear as animals lack the metabolic pathways to metabolize it. Some studies have observed that herbicide, such as Atrazine, can disrupt the endocrine and reproductive systems. Pollinators, such as honeybees, are exposed to significant concentrations of pesticides in the field, especially the most commonly used family of herbicides, Glyphosates.

One of the first lines of defense against pesticides exposure is the gut microbiome; however, the way it can impact toxicity of pesticides currently remains unknown. Some naturally occurring bacteria can metabolize Atrazine into compounds such as cyanuric acid, biuret, and allophanate—which have toxicity profiles different from Atrazine. While certain gut microbes are known to convey fitness advantages, dysbiosis (e.g., microbial imbalance) can lead to damaging effects, diseases, and immune system failure. Given the importance of pollinators and the associated risk to human health, studying the effects of Atrazine and Glyphosate exposure on the microbiome and whether it leads to dysbiosis that impacts health, productivity, and survival.

### SUMMARY

Work described herein has shown that two bacteria, S. marcescens and P. protegens, are useful in preventing the toxic effects of an herbicide (e.g., atrazine or glyphosate) in a subject following exposure (e.g., ingesting) to the herbicide. These strains, identified herein as S. marcescens strain NVIT01 and P. protegens strain NVIT02, alter the gut microbiome in a manner that resulted in the detoxification of the herbicides. Subject with this altered gut microbiome were found to be resistant to further exposures of the herbicide. Additionally, work described herein shows that the altered gut microbiome was found in offspring of subjects who were previously administered the bacteria, showing that the altered gut microbiome is inherited.

Accordingly, one aspect of the invention described herein provides a composition for detoxifying an herbicide comprising viable bacteria of the Serratia genus and/or viable bacteria of the Pseudomonas genus and a carrier.

In one embodiment of any aspect, the composition for detoxifying an herbicide comprises viable Serratia marcescens (S. marcescens) bacteria and a carrier. In another embodiment of any aspect, the viable bacteria of the Serratia genus is Serratia marcescens (S. marcescens).

In one embodiment of any aspect, the S. marcescens strain is NVIT01.

In one embodiment of any aspect, the S. marcescens is present in the composition at an abundance of at least 3×10⁸ cells/mL.

In one embodiment of any aspect, the composition for detoxifying an herbicide comprises viable Pseudomonas protegens (P. protegens) bacteria and a carrier.

In one embodiment of any aspect, the P. protegens strain is NVIT02.

In one embodiment of any aspect, the P. protegens is present in the composition at an abundance of at least 8×10⁶ cells/mL.

In one embodiment of any aspect, the composition for detoxifying an herbicide, comprises a mixture of viable bacteria including S. marcescens, P. protegens, and a carrier.

In one embodiment of any aspect, the carrier is phosphate-buffered saline. In one embodiment of any aspect, the carrier promotes bacterial growth.

In one embodiment of any aspect, the composition does not elicit an immune response.

In one embodiment of any aspect, the composition is formulated for aerosol delivery.

In one embodiment of any aspect, the composition is formulated for oral delivery.

A second aspect of the invention described herein provides a method of detoxifying an herbicide consumed by a subject comprising administering any of the compositions described herein to a subject exposed to an herbicide.

In one embodiment of any aspect, the subject is an insect or a mammal.

In one embodiment of any aspect, the subject is human.

In one embodiment of any aspect, the herbicide is atrazine or glyphosate.

In one embodiment of any aspect, the administering is an aerosol administration. In one embodiment of any aspect, the administering is an oral administration. In one embodiment of any aspect, the administering the compositions detoxifies the herbicide.

A third aspect of the invention described herein provides a method of promoting a resistance to an herbicide comprising administering any of the compositions described herein to a subject in need thereof.

A fourth aspect of the invention described herein provides a method of treating a body of water comprising administering any of the compositions described herein to a body of water.

In one embodiment of any aspect, the body of water is a municipal water supply.

In one embodiment of any aspect, the body of water contains an herbicide.

### Definitions

As used herein, “detoxify” refers to the process of removing a harmful effects (e.g., the toxic action) or an herbicide. An herbicide can be “detoxified”, e.g., via its complete or partial degradation to a non-harmful by-product. As used herein, “resistance” refers to the acquired capacity to detoxify an herbicide.

As used herein, the term “does not elicit an immune response,” and equivalent terms “does not induce an immune response” and “does not promote an immune response” or similar variations thereof mean that a given microbial species, e.g., a bacterial species, does not substantially induce inflammatory response by the host immune system. A microbe that does not induce an immune response will not, when administered to a subject, promote an expression or accumulation of inflammatory chemokines or cytokines, to significantly increased levels above those of a healthy unperturbed baseline or the recruitment or accumulation of inflammatory host cells.

The terms “increased”, or “increase”, are all used herein to mean an increase by a statistically significant amount. In some embodiments, the terms “increased”, or “increase”, can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The terms “lower”, “reduced”, “reduction” or “decrease”, “down-regulate” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “statistically significant” or “significantly” refer to statistical significance and generally means a two standard deviation (2SD) or greater difference.

The term “carrier” as used herein means acceptable material, composition or vehicle, such as a liquid or solid filler, diluent, excipient, solvent or encapsulating material, involved in carrying or transporting the active ingredient (e.g., bacteria) to the targeting place in the body of a subject. Each carrier must be “acceptable” in the sense of being compatible with the other ingredients of the formulation and is compatible with administration to a subject, for example a human. In one embodiment, the carrier is something other than water or cell culture media. Carriers known in the art can include serum proteins, human serum albumin, liposomes, buffers such as phosphates, water, sterile saline or other salts, phosphate-buffered saline, electrolytes, glycerol, hydroxymethylcellulose, propylene glycol, polyethylene glycol, polyoxyethylenesorbitan, other surface active agents, vegetable oils, and conventional anti-bacterial (e.g., to prevent growth of other bacteria that is not useful in the invention described herein) or anti-fungal agents, such as parabens, chlorobutanol, phenol, sorbic acid, thimerosal, and the like. A carrier use herein will promote the growth of a bacteria described herein to be helpful in detoxifying an herbicide, for example, S. marcescens and/or P. protegens.

As used herein, a “subject” means a human, animal, or insect. Usually the animal is a vertebrate such as a primate, rodent, domestic animal, livestock, farm animal, or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox or wolf. In some embodiments, the insect is a pollinating insects. Non-limiting examples of pollinating insects include bees, (e.g., honey bees, solitary species, bumblebees), pollen wasps (e.g., Masarinae), ants, flies (e.g., bee flies, hoverflies, mosquitoes), lepidopterans (e.g., butterflies and moths), and flower beetles. In some embodiments, the subject is a mammal, e.g., a primate, e.g., a human.

A subject can have been previously exposed to an herbicide, not been previously exposed to an herbicide, or is at risk of being exposed to an herbicide (e.g., a subject who works with an herbicide, e.g., a farm worker).

As used herein, the terms “treat,” “treatment,” “treating,” or “amelioration” refer to therapeutic treatments, wherein the object is to reverse, inhibit, or remove the toxicity of an herbicide (e.g., an atrazine or glyphosate) from the source it is contained in (e.g., a body of water, or a subject). Treatment is generally “effective” if the toxicity of at least an herbicide is reduced. A “treatment” can promote resistance to the toxicity of future herbicide exposure in the source. Treatment can, but does not necessarily, include 100% eradication of the toxicity of at least an herbicide—a treatment that reduces the level of toxicity of at least an herbicide contained in a source is considered effective as the term is used herein.

The term “administered” refer to a subject being treated with an effective dose of any composition described herein by methods that deliver the composition, e.g., aerosol or oral administration.

The term “optional” or “optionally” means that the subsequent described event, circumstance or substituent may or may not occur, and that the description includes instances where the event or circumstance occurs and instances where it does not.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIGS. 1A-1D present data showing effects of S. marcescens and P. protegens on atrazine. (FIG. 1A) A single colony starting inoculum of each bacterium, S. marcescens NVIT01 (48 hr) and (FIG. 1B) P. protegens NVIT02 (72 hr) were incubated in minimal growth media containing 30 ppb atrazine at 28° C. An aliquot of 100 μl of the culture media was plated on LB media and cultures counted after 24 hr incubation at 28° C. for Colony-Forming Units (CFU) (Two-tailed t test, *P<0.05, **P<0.01, ns=not significant). (FIG. 1C) Zones of clearing (darker halo around bacterial streaks) by S. marcescens NVIT01 and (FIG. 1D) P. protegens NVIT02 on LB agar plates with atrazine dense matrix, white haze around bacterial streaks (300 mg/ml).

FIGS. 2A-2C present data that show bacterial density in the naïve and atrazine exposed populations. Quantitative PCR analysis of (FIG. 2A) Nasonia host DNA, S6K, or total bacterial load, 16S. Relative abundance of (FIG. 2B) S. marcescens NVIT01 and (FIG. 2C) P. protegens NVIT02 densities relative to Nasonia host DNA, S6K. (Unpaired t test, *P<0.05).

FIGS. 3A-3C present data that show atrazine resistance. (FIG. 3A) The median lethal concentration of atrazine to kill half of the population (LC50) in ppm for each generation of the Naïve populations (open circles) and the Atrazine exposed populations (black circles). (FIG. 3B) The median lethal concentration of atrazine to kill half of the population (LC50) in ppm for each generation of the Naïve populations (open circles) and the Atrazine exposed populations (black circles) when grown Germfree, gnotobiotic. Without bacteria, both populations experience increased toxicity of atrazine, over 100 fold greater than normal generations' LC50. (FIG. 3C) LC50 survival of 24 hrs old adult Nasonia virtipennis were sprayed with a sterile PBS solution (squares), S. marcescens TVIT01 (8×10⁶ cells/ml, circles), or P. protegens TVIT02 (3×10⁸ cells/ml, triangles) and incubated for 6 hr before being fed 100 ppm of atrazine sugar solution (A) or only sugar solution (C), and their survival was calculated 48 hrs later. Three replicates of 50 individuals are for each treated group (Two-tailed t test, *P<0.05).

### DETAILED DESCRIPTION

Compositions and methods are described herein for detoxifying or promoting resistance to an herbicide. The compositions and methods are based, in part, upon the identification of a two bacteria, S. marcescens and P. protegens, that are capable of preventing the toxic effects of an herbicide (e.g., atrazine or glyphosate) in a subject. Specifically, the S. marcescens strain NVIT01 and P. protegens strain NVIT02 are used herein. It was found that, upon administration of these two strains of bacteria, the gut microbiome of the subject was altered. The presence of these bacteria in the gut facilitated the detoxification of the herbicides as it was consumed by the subject, e.g., the subjects were now resistant to exposure of the herbicide. Surprisingly, this altered gut microbiome was found to be hereditary; these bacteria were found in offspring of subjects who had been previously administered the bacteria.

Further, described herein is a method for treating a body of water (e.g., a water supply source) with S. marcescens and/or P. protegens to detoxify the herbicide prior to consumption by a subject.

Various considerations useful for the practice of the compositions and methods disclosed herein are set out in the following.

### Serratia Species

Serratia is a genus of gram-negative, facultative anaerobic, rod-shaped bacteria from the Enterobacteriaceae family. The most common species of this genus is S. marcescens, which can be distinguished from other members of the Enterobacteriaceae family via its unique expression of three enzymes—DNase, lipase, and gelatinase. Expression of these three enzymes can be assessed by a skilled person via, e.g., western blotting or PCR-based assays to measure the protein or mRNA levels, respectively. S. marcescens can also be identified by, e.g., visualizing its production of the red pigment, prodigiosin.

S. marcescens is an opportunistic human pathogen, though infections caused by this bacterium are often acquired in a hospital setting. Infections caused by S. marcescens are most often due to colonization of the bacteria in the respiratory or urinary tracts of a subject, rather than the gastrointestinal tract. In one embodiment, administration of a composition comprising S. marcescens does not cause an infection in the subject.

In one embodiment, the S. marcescens strain is NVIT01. The 16S rDNA sequence of S. marcescens strain NVIT01 is as follows:

(SEQ ID NO: 1)
TCCCGAAGGTTAAGCTACCTACTTCTTTTGCAACCCACTCCCATGGTGTG
ACGGGCGGTGTGTACAAGGCCCGGGAACGTATTCACCGTAGCATTCTGAT
CTACGATTACTAGCGATTCCGACTTCATGGAGTCGAGTTGCAGACTCCAA
TCCGGACTACGACATACTTTATGAGGTCCGCTTGCTCTCGCGAGGTCGCT
TCTCTTTGTATATGCCATTGTAGCACGTGTGTAGCCCTACTCGTAAGGGC
CATGATGACTTGACGTCATCCCCACCTTCCTCCAGTTTATCACTGGCAGT
CTCCTTTGAGTTCCCGGCCGAACCGCTGGCAACAAAGGATAAGGGTTGCG
CTCGTTGCGGGACTTAACCCAACATTTCACAACACGAGCTGACGACAGCC
ATGCAGCACCTGTCTCAGAGTTCCCGAAGGCACCAATCCATCTCTGGAAA
GTTCTCTGGATGTCAAGAGTAGGTAAGGTTCTTCGCGTTGCATCGAATTA
AACCACATGCTCCACCGCTTGTGCGGGCCCCCGTCAATTCATTTGAGTTT
TAACCTTGCGGCCGTACTCCCCAGGCGGTCGATTTAACGCGTTAGCTCCG
GAAGCCACGCCTCAAGGGCACAACCTCCAAATCGACATCGTTTACAGCGT
GGACTACCAGGGTATCTAATCCTGTTTGCTCCCCACGCTTTCGCACCTGA
GCGTCAGTCTTCGTCCAGGGGGCCGCCTTCGCCACCGGTATTCCTCCAGA
TCTCTACGCATTTCACCGCTACACCTGGAATTCTACCCCCCTCTACGAGA
CTCTAGCTTGCCAGTTTCAAATGCAGTTCCCAGGTTGAGCCCGGGGATTT
CACATCTGACTTAACAAACCGCCTGCGTGCGCTTTACGCCCAGTAATTCC
GATTAACGCTTGCACCCTCCGTATTACCGCGGCTGCTGGCACGGAGTTAG
CCGGTGCTTCTTCTGCGAGTAACGTCAATTGATGAACGTATTAAGTTCAC
CACCTTCCTCCTCGCTGAAAGTGCTTTACAACCCGAAGGCCTTCTTCACA
CACGCGGCATGGCTGCATCAGGCTTGCGCCCATTGTGCAATATTCCCCAC
TGCTGCCTCCCGTAGGAGTCTGGACCGTGTCTCAGTTCCAGTGTGGCTGG
TCATCCTCTCAGACCAGCTAGGGATCGTCGCCTAGGTGAGCCATTACCCC
ACCTACTAGCTAATCCCATCTGGGCACATCTGATGGCAAGAGGCCCGAAG
GTCCCCCTCTTTGGTCTTGCGACGTTATGCGGTATTAGCTACCGTTTCCA
GTAGTTATCCCCCTCCATCAGGCAGTTTCCCAGACATTACTCACCCGTCC
GCCGCTCGTCACCCAGGGAGCAAGCT

S. marcescens strain NVIT0116S rRNA gene sequence (SEQ ID NO: 1) can be found, e.g., GenBank ID MG426189-MG426190.

Pseudomonas Species

Pseudomonas is a gram-negative, rod shaped, aerobic, non-spore forming, Gammaproteobacteria that belongs to the family Pseudomonadaceae. Presently, there are 191 validated species of Pseudomonas. A skilled person can identify a species of Pseudomonas, e.g., via a positive oxidase test result, the absence of gas formation from glucose, a negative methyl red result, and/or a negative Voges-Proskauer test.

P. protegens is a typical soil microorganism, that provides protection to plants. P. protegens produces the antimicrobial compounds pyoluteorin and 2,4-diacetylphloroglucinol (DAPG), which are active against various plant pathogens. A skilled person can identify a strain of P. protegens via assessing its known characteristics, e.g., expression of the antimicrobial compounds described herein, aerobe positive test, oxidase positive test, and the presence of 1 to 3 flagella.

In one embodiment, the P. protegens strain is NVIT02. The 16S rDNA sequence of P. protegens strain NVIT02 is as follows:

GCGGCGGACGGGTGAGTAATGCCTAGGAATCTGCCTAGTAGTGGGGGATA
ACGTCCGGAAACGGGCGCTAATACCGCATACGTCCTACGGGAGAAAGTGG
GGGATCTTCGGACCTCACGCTATTAGATGAGCCTAGGTCGGATTAGCTAG
TTGGTGAGGTAATGGCTCACCAAGGCGACGATCCGTAACTGGTCTGAGAG
GATGATCAGTCACACTGGAACTGAGACACGGTCCAGACTCCTACGGGAGG
CAGCAGTGGGGAATATTGGACAATGGGCGAAAGCCTGATCCAGCCATGCC
GCGTGTGTGAAGAAGGTCTTCGGATTGTAAAGCACTTTAAGTTGGGAGGA
AGGGCAGTTACCTAATACGTGATTGTTTTGACGTTACCGACAGAATAAGC
ACCGGCTAACTCTGTGCCAGCAGCCGCGGTAATACAGAGGGTGCAAGCGT
TAATCGGAATTACTGGGCGTAAAGCGCGCGTAGGTGGTTTGTTAAGTTGG
ATGTGAAAGCCCCGGGCTCAACCTGGGAACTGCATCCAAAACTGGCAAGC
TAGAGTATGGTAGAGGGTGGTGGAATTTCCTGTGTAGCGGTGAAATGCGT
AGATATAGGAAGGAACACCAGTGGCGAAGGCGACCACCTGGACTGATACT
GACACTGAGGTGCGAAAGCGTGGGGAGCAAACAGGATTAGATACCCTGGT
AGTCCACGCCGTAAACGATGTCAACTAGCCGTTGGGAGCCTTGAGCTCTT
AGTGGCGCAGCTAACGCATTAAGTTGACCGCCTGGGGAGTACGGCCGCAA
GGTTAAAACTCAAATGAATTGACGGGGGCCCGCACAAGCGGTGGAGCATG
TGGTTTAATTCGAAGCAACGCGAAGAACCTTACCAGGCCTTGACATCCAA
TGAACTTTCTAGAGATAGATTGGTGCCTTCGGGAACATTGAGACAGGTGC
TGCATGGCTGTCGTCAGCTCGTGTCGTGAGATGTTGGGTTAAGTCCCGTA
ACGAGCGCAACCCTTGTCCTTAGTTACCAGCACGTTATGGTGGGCACTCT
AAGGAGACTGCCGGTGACAAACCGGAGGAAGGTGGGGATGACGTCAAGTC
ATCATGGCCCTTACGGCCTGGGCTACACACGTGCTACAATGGTCGGTACA
AAGGGTTGCCAAGCCGCGAGGTGGAGCTAATCCCATAAAACCGATCGTAG
TCCGGATCGCAGTCTGCAACTCGACTGCGTGAAGTCGGAATCGCTAGTAA
TCGCGAATCAGAATGTCGCGGTGAATACGTTCCCGGGCCTTGTACACACC
GCCC

P. protegens strain NVIT02 16S rRNA gene sequence (SEQ ID NO: 2) can be found, e.g., GenBank ID MG592715-MG592717.

### Compositions for Detoxification of an Herbicide

One aspect of the invention described herein provides a composition for detoxifying an herbicide comprising viable Serratia marcescens (S. marcescens) bacteria and a carrier.

Another aspect of the invention described herein provides composition for detoxifying an herbicide comprising viable Pseudomonas protegens (P. protegens) bacteria and a carrier.

In one embodiment, the S. marcescens strain is NVIT01. In another embodiment, the S. marcescens strain is a S. marcescens strain having an 16S rRNA comprising at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to the 16S rRNA of S. marcescens strain NVIT01 (e.g., SEQ ID NO: 1), and comprises the detoxification activity of S. marcescens strain NVIT01. In one embodiment, the S. marcescens strain comprises at least 50%, at least 60%, at least 70%, at least 80%, at least 90%, at least 95%, at least 99%, or more of the detoxification activity of S. marcescens strain NVIT01.

In one embodiment, the composition comprises a concentration of S. marcescens bacteria that is at least 3×10⁸ cells/mL. In one embodiment, the composition comprises a concentration of S. marcescens bacteria that is between 3×10⁸ cells/mL and 3×10⁹ cells/mL.

In one embodiment, the P. protegens strain is NVIT02. In another embodiment, the P. protegens strain is a P. protegens strain having an 16S rRNA comprising at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to the 16S rRNA of P. protegens strain NVIT02 (e.g., SEQ ID NO: 2), and comprises the detoxification activity of P. protegens strain NVIT02. In one embodiment, the P. protegens strain comprises at least 50%, at least 60%, at least 70%, at least 80%, at least 90%, at least 95%, at least 99%, or more of the detoxification activity of P. protegens strain NVIT02

In one embodiment, the composition comprises a concentration of P. protegens bacteria that is at least 8×10⁶ cells/mL. In one embodiment, the composition comprises a concentration of P. protegens bacteria that is between 8×10⁶ cells/mL and 8×10⁹ cells/mL.

One aspect of the invention described herein is a composition comprising a mixture of viable bacteria consisting of S. marcescens, P. protegens, and a carrier. In one embodiment, the ratio of viable bacteria in the composition is important for its efficacy.

In one embodiment, the ratio of viable S. marcescens bacteria to viable P. protegens bacteria in the composition is 1:1, 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, or 9:10.

In one embodiment, the ratio of viable P. protegens bacteria to viable S. marcescens bacteria in the composition is 1:1, 1:2, 1:3, 1:4, 1:5, 1:6, 1:7, 1:8, 1:9, 1:10, 2:3, 2:5, 2:7, 2:9, 3:4, 3:5, 3:7, 3:8, 3:10, 4:5, 4:7, 4:9, 5:6, 5:7, 5:8, 5:9, 6:7, 7:8, 7:9, 7:10, 8:9, or 9:10.

The above described bacteria comprised in the compositions include, for example but not limited to, viable bacteria, wet bacteria, dry viable bacteria (e.g., preparations including viable spray-dried cells, freeze-dried cells, vacuum-dried cells, drum-dried cells, vitrified etc.), and the like. Preparations of bacteria species described herein (e.g., S. marcescens and P. protegens) can include, for example, suspensions of bacteria, cultured cells of bacteria (including bacterial cells, supernatant, and medium ingredients), and cultured media containing bacteria (obtained by removing solid contents from the cultured cells of bacteria). While viable bacteria are used in most applications considered herein, it is contemplated that in some embodiments, processed cells of a given bacteria (e.g., S. marcescens and P. protegens) can include, for example, ground cells, crushed cells, liquefied cells (extracts etc.), concentrates, paste-like cells, dried preparations thereof, and the like.

In one embodiment, the bacteria comprised by the composition are lyophilized, or freeze-dried in a manner that preserves bacterial viability. Methods of preserving viable bacteria by lyophilization can promote long-term preservation of the microorganism. One skilled in the art will be able to lyophilize bacteria using standard techniques. Briefly, microbes are cultured and suspended in lyophilizing buffer or medium. The microbes are rapidly frozen and then subjected to a primary and secondary drying phase to remove all readily available water and residual water, respectively. Storage at 4° C. or lower is recommended, with no moisture present. Standard bacterial lyophilizing techniques can be found in, for example, Perry, S. F., Cryopreservation and freeze-drying protocols. Volume 38, pg 21-30.

In some aspects, the compositions describe herein comprise a carrier. In one embodiment, the carrier is a phosphate-buffered saline. In some embodiments, carrier promotes bacterial growth of the bacteria useful for the detoxification of an herbicide (e.g., S. marcescens and/or P. protegens). Non-limiting examples of carriers that promote bacterial growth include boric acid, a prebiotic, and low pH buffering agents as sodium bicarbonate and agents that can act as both acidifying and prebiotics, such as ascorbic acid (vitamin C). A carrier described herein can make up at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more of a composition described herein.

In one embodiment, the composition further comprises at least one excipient. Excipients are substances added to an active ingredient for the purpose of long-term stabilization, bulking up solid formulations that contain active ingredients (known as “bulking agents”), or to enhance or protect the therapeutic benefit of an active ingredient by facilitating drug absorption, reducing viscosity, or increasing solubility or reconstitution from a dry state. Non-limiting examples of protective excipients include a nonreducing monosaccharide, sugar alcohol, oligosaccharide, amino acid, polyvinylpyrrolodone, polyethylene glycol, Ficol, inulin, albumin, gelatin, whey proteins, and/or a polaxomer. In some embodiments, the composition further comprises at least one, at least two, at least three, at least four, or at least five or more protective excipients. An excipient described herein can make up at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or more of a composition described herein.

In one embodiment, an effective amount of viable S. marcescens bacteria and/or viable P. protegens is an amount that is effective at detoxifying an herbicide (e.g., atrazine) and does not cause an infection in the subject, e.g., a human.

In one embodiment, viable S. marcescens and/or viable P. protegens bacteria do not exhibit pathogenicity. In one embodiment, viable S. marcescens and/or viable P. protegens bacteria are modified such that they lack their pathogenicity. As used herein, a “virulence factor” refers to a molecule produced by a bacterium that aides in the pathogenicity of the bacterium, for example, colonization in a host, evasion of the host's immune system, inhibition of the host's immune system, entry into or exit of a cell, and/or obtaining nutrition from a host. A skilled practitioner can identify the virulence factors for each bacteria and modify the bug such that it lacks such factors. At a minimum, the bacteria are modified such that they are unable to infect a subject, e.g., in the respiratory system, or urinary tract. For example, one could select for a S. marcescens strain which exhibit none or the least amount of pathogenic traits, or one could genetically deplete the virulence factors from the genome, e.g., using gene editing tools, such as CRISPR.

In one embodiment, the composition can be formulated for aerosol delivery. For use as aerosols, a composition described herein can be provided in solution or suspension and may be packaged in a pressurized aerosol container together with suitable propellants, for example, hydrocarbon propellants like propane, butane, or isobutane with conventional adjuvants.

In one embodiment, the composition can be formulated for controlled- or extended-release. Controlled-release pharmaceutical products have a common goal of improving drug therapy over that achieved by their non-controlled release counterparts. Ideally, the use of an optimally designed controlled-release preparation in medical treatment is characterized by a minimum of drug or active substance being employed to cure or control the condition in a minimum amount of time. Advantages of controlled-release formulations include: 1) extended activity of the drug or active substance; 2) reduced dosage frequency; 3) increased patient compliance; 4) usage of less total drug or active substance; 5) reduction in local or systemic side effects; 6) reduction in blood level fluctuations, where appropriate; 7) improvement in efficacy of treatment; 8) reduction of potentiation or loss of drug activity; and 9) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug or active ingredient (e.g., the bacteria described herein) that promptly produces the desired therapeutic effect, and gradually and continually release other amounts of drug or active ingredient to maintain this level of therapeutic or prophylactic effect over an extended period of time. In order to maintain this constant level, the drug or active ingredient must be released from the dosage form at a rate that will replace the amount of drug or active ingredient being metabolized or otherwise lost from the body. Controlled-release of an active ingredient can be stimulated by various conditions including, but not limited to, pH, ionic strength, osmotic pressure, temperature, enzymes, water, and other physiological conditions or compounds.

A variety of known extended-release dosage forms, formulations, and devices can be adapted for use with viable bacterial compositions described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

### Detoxifying an Herbicide

In one aspect described herein, any of the compositions described herein are administered to a subject who has been exposed to an herbicide to detoxify the herbicide. In one aspect described herein, any of the compositions described herein are administered to a subject who has not been exposed to an herbicide. In one aspect described herein, any of the compositions described herein are administered to a subject who will be exposed to an herbicide in the future following administration.

An herbicide is a chemical substance used to control plant growth, and is common in agriculture. Herbicides can be applied e.g., directly to the soil or applied to the portion of the plant that is above ground resulting in it being absorbed into the plant tissue. Exposure to an herbicide can be indirect, e.g., through contact or consumption of food or water that was contacted with an herbicide.

An herbicide is classified by its mechanism of action. Non-limiting examples of classifications of herbicides include Acetyl CoA Carboxylase (ACCase) inhibitors, acetolactate (ALS) inhibitors, 5-enolpyruvylshikimate-3-phosphate (EPSP) synthase inhibitors, Synthetic auxins, Photosystem II inhibitors, Photosystem I inhibitors, and 4-Hydroxyphenylpyruvate dioxygenase (HPPD) inhibitors.

Atrazine, a common herbicide, is a trizine class member. Atrazine is the most commonly found herbicide in the drinking water systems in the United States, and it has been suggested that this herbicide can result in a hormonal imbalance following its consumption by, or contact with, humans. Atrazine can be degraded (e.g., detoxified) via two mechanisms. 1) Hydrolysis of the C—Cl bond is followed by the ethyl and isopropyl groups, catalyzed by the hydrolase enzymes called AtzA, AtzB, and AtzC. The end product of this process is cyanuric acid, itself unstable with respect to ammonia and carbon dioxide. 2) Dealkylation of the amino groups gives 2-chloro-4-hydroxy-6-amino-1,3,5-triazine, the degradation of which is unknown.

In one embodiment, detoxification of atrazine results in the by-product cyanuric acid. In one embodiment, detoxification of atrazine results in the by-product 2-chloro-4-hydroxy-6-amino-1,3,5-triazine.

Glyphosate (N-(phosphonomethyl)glycine) is a broad-spectrum systemic herbicide and crop desiccant. Glyphosate became a popular herbicide when crops that were resistant to Glyphosate were introduced. This herbicide is applied directly to the plant and is absorbed into the plant tissue and root, and it trafficked to the sites of growth. Glyphosate is readily degraded to aminomethylphosphonic acid (AMPA), a weak organic acid with a phosphonic acid group. AMPA can be further degraded to phosphoric acid, and then to carbon dioxide and inorganic phosphate.

In one embodiment, detoxification of Glyphosate results in the by-product AMPA. In one embodiment, detoxification of Glyphosate results in the by-product phosphoric acid. In one embodiment, detoxification of Glyphosate results in the by-product carbon dioxide. In one embodiment, detoxification of Glyphosate results in the by-product inorganic phosphate.

### Promoting Resistance to an Herbicide

In one embodiment, a composition described herein is administered to a subject to promote resistance to toxicity of an herbicide. As used herein, “resistance” refers to the acquired ability to degrade, e.g., detoxify, an herbicide, whereas the subject could not degrade the herbicide prior to acquiring its resistance. A composition's capacity to promote resistance can be determined, e.g., by assessing whether a subject who has been administered a composition described herein can, e.g., survive a second exposure to the herbicide. For example, a honey bee is unable to survive exposure to atrazine. If a honey bee is able to survive exposure to atrazine following administration of a composition described herein, it can be determined that the honey bee is now resistant to the herbicide (e.g., atrazine).

In one embodiment, administration of any of the compositions described herein results in an alteration of the gut microbiome, such that these bacteria are present at an increased level as compared to prior to administration. In one embodiment, administration of any of the compositions described herein results in an increase in the bacteria's abundance in the gut microbiome by 2-fold or more as compared to the abundance of the bacteria prior to administration. The abundance of a given bacteria in the gut microbiome of a subject can be assessed, e.g., by sequencing 16S rRNA in fecal sample obtain from a subject, e.g., prior to and following administration of a composition described herein. In one embodiment, the establishment of a population of the bacteria in the gut microbiome in a subject promotes resistance to an herbicide. The established bacterial population can, for example, metabolize or breakdown an herbicide that the subject will come in contact with in the future following administration of the composition.

In one embodiment, the abundance of a given bacteria (e.g., S. marcescens, or P. protegens) in the gut microbiome of a subject is passed on to its offspring.

### Treating Bodies of Water

Herbicides have been commonly found in drinking supply systems in the United States, resulting in the unintentional mass consumption of herbicides. In one aspect of the invention is a method of treating a body of water comprising administering any of the compositions described herein to a body of water. In one embodiment, the administration results in the presence of a given bacteria (e.g., S. marcescens, or P. protegens) sufficient to degrade (e.g., detoxify) the presence of any herbicide in the water source. In one embodiment, the administration results in the presence of a given bacteria (e.g., S. marcescens, or P. protegens) sufficient to degrade (e.g., detoxify) any herbicide that will be exposed to the water source.

In one embodiment the body of water is exposed to an herbicide. Non-limiting mechanisms of exposure include rain run-off from plants treated with herbicides, aerosol treatment of herbicides, absorption of an herbicide in ground water.

In one embodiment, the body of water is a municipal water supply. In one embodiment, the body of water is a rural water supply. In one embodiment, the body of water is a well. In one embodiment, the body of water is a lake. In one embodiment, the body of water is a pond (e.g., aquaculture ponds or freshwater ponds). In one embodiment, the body of water is a stream. In one embodiment, the body of water is a water retention system (e.g., a rain retention drum) or a urban drainage system. In one embodiment, the water source is a source of water consumed by or exposed to a subject described herein (e.g., a honeybee, a livestock animal, or a human).

### Dosages Forms and Administration

The dosages of compositions comprising a bacterial mixture that detoxify or promote resistance to an herbicide can be determined by one of ordinary skill e.g., by assessing the effects of the toxicity of a given herbicide. The interrelationship of dosages for animals of various sizes and species and humans based on mg/m³ of surface area is described by E. J. Freireich et al., “Quantitative Comparison of Toxicity of Anticancer Agents in Mouse, Rat, Hamster, Dog, Monkey and Man,” Cancer Chemother. Rep. 50: 219-244 (1966). Adjustments in the dosage regimen can be made to optimize the therapeutic response. Doses can be divided and administered on a daily basis or the dose can be reduced proportionally depending on the therapeutic situation.

In some embodiments, the composition comprising a bacteria or a bacterial mixture to detoxify, or promote resistance to, an herbicide is administered to a subject who has been exposed to or will be exposed to an herbicide. Microorganisms and/or spores can be separated and selected, using any one of a number of methods that are well known to those of ordinary skill in the art, for their bioactive properties to help detoxify or promote resistance to an herbicide. An effective amount of microorganisms and/or their spores is an amount sufficient to detoxify or promote resistance to an herbicide. In accordance with these embodiments, an effective amount of microorganisms is from 100 thousand to 500 thousand, from 500 thousand to 1 million, from 1 million to 50 million, from 50 million to 100 million, from 100 million to 500 million, from 500 million to 1 billion, from 1 billion to 50 billion, from 50 billion to 100 billion, from 100 billion to 500 billion, from 500 billion to 600 billion CFU per dose, where the dose is administered, for example, daily, weekly, monthly, yearly, one or more times per week, or as often as about one to three times daily.

The dosage range depends upon the potency, and includes amounts large enough to produce the desired effect, e.g., detoxify or promote resistance to an herbicide. The dosage should not be so large as to cause unacceptable adverse side effects, e.g., a respiratory infection. Generally, the dosage will vary with the type of agent (e.g., bacteria), and with the type of subject (e.g., a human, honey bee, or livestock). The dosage can be determined by one of skill in the art and can also be adjusted by the individual physician in the event of any complication. Typically, the dosage will range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain levels between 5 μg/mL and 30 μg/mL, for example in the gut of the subject.

The means by which the composition comprising the bacterial compositions described herein should be administered should be appropriate for the given composition. In one embodiment the composition will be administered orally. Microorganisms can be administered in a suspension in liquid form, in a slurry, in a capsule, or, for example, in dried form in a capsule. Methods for maintaining viability of microorganisms throughout the drying process are known to those of skill in the art. Microorganisms, including, but not limited to dried preparations, can also be formulated in enteric-coated or other forms such that when administered orally the microorganisms avoid killing in the harsh acidic conditions of the stomach and are only released to re-hydrate/reactivate in the relatively safer environment of the intestine. Microorganisms can also be administered in admixture with a food or beverage product, including, but not limited to a yogurt, kefir or other dairy product, or as dried microbes in, for example, a bar of cereal, granola, cattle feed, etc. Microorganisms useful in the methods and compositions described herein can also be prepared and/or administered in admixture with one or more prebiotic compositions that promote the maintenance, establishment and/or growth of the probiotic.

Composition comprising a bacterial mixture for the detoxification of, or promotion of resistance to, an herbicide can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

### Efficacy Measurement

The efficacy of a given composition can be determined by the skilled clinician. However, a treatment is considered “effective treatment,” as the term is used herein, if the toxicity of an herbicide is reduced, e.g., by at least 10% following administration of a composition comprising a bacterial mixture described herein. Efficacy can also be measured by resistance to an herbicide (e.g., to degrade an herbicide a subject is exposed to following administration). Methods of measuring these indicators are known to those of skill in the art and/or described herein.

Effective amounts, toxicity, and therapeutic efficacy of drug agents, e.g., bacteria, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from in vivo assays. Also, a dose can be formulated in animal models to achieve a concentration range that includes the IC₅₀ (i.e., the concentration of the active ingredient, which achieves a half-maximal inhibition of symptoms). Levels in the subject can be measured, for example, by high performance liquid chromatography or other appropriate technique. The effects of any particular dosage can be monitored by a suitable bioassay. The dosage can be determined by a physician and adjusted, as necessary, to suit observed effects of the treatment.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Examples

Data presented herein show the application of bacterial probiotic (e.g., Serratia marcescens and/or Pseudomonas protegens) as a detoxicant to chemical xenobiotic exposure for honeybees, livestock, humans and drinking water.

Agrochemicals, such as insecticides and herbicides, are valuable for producing larger amounts of food, but their global use comes with risk to the insect pollinators, the farmers that use them, drinking water and food consumed by a subject described herein. Although there are risk assessments of the non-target effects on environmental and human health these compounds can impose, one aspect of biology that is not understood is the influence of the microbiome can have on detoxifying these compounds. Described herein are experiments using the model insect system Nasonia, the standard animal model most closely related to the honeybee, to observe laboratory populations that are naïve and exposed to the herbicide Atrazine. Soil and water bacteria have previously been identified as being able to metabolize the compound, but no observations are recorded in gut microbiomes or their impact on the toxicity. From these populations of animals, two bacterial isolates were identified as having the metabolic capacity to convert agrochemical xenobiotics, including Atrazine and Glyphosate families of compounds, into less toxic forms. Furthermore, when the isolated bacteria were applied as probiotics to naïve animals an increased survival was observed as the bacteria act as a detoxicant to the host animal.

Identification of Bacterial Isolates

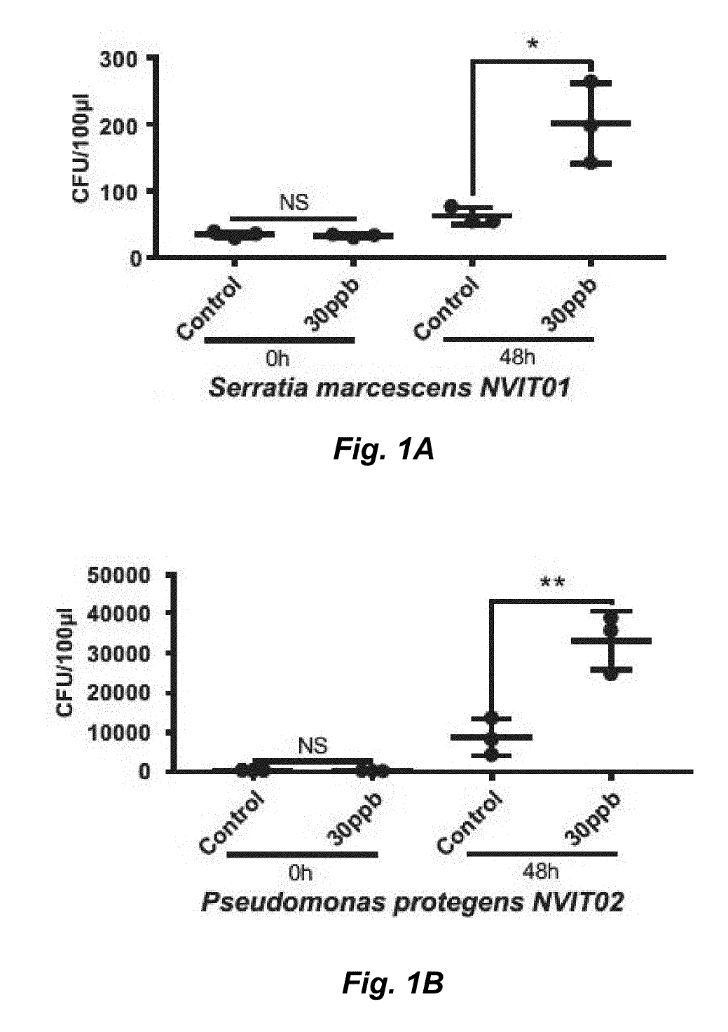

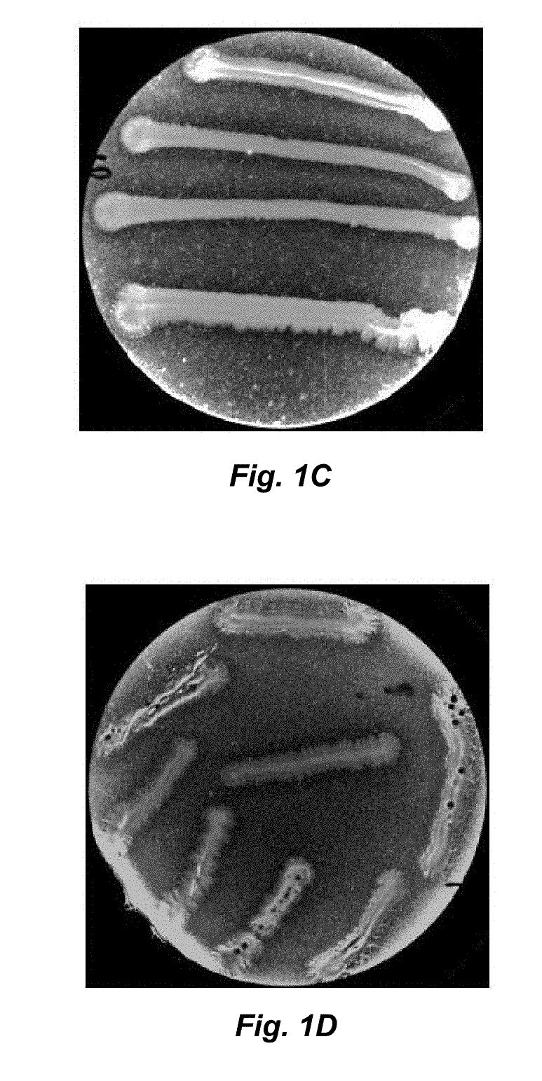

Bacterial isolates that are present in population of animals habitually exposed to nonlethal concentrations of atrazine (30 ppb) for 32 generations were observed. By homogenizing animal gut tissue in minimal media containing Atrazine at 30 ppb and serially passaging the cultures two bacterial isolates with significant metabolic capacity to degrade Atrazine were identified: Serratia marcescens NVIT01 and Pseudomonas protegens NVIT02. Both bacteria produced an increased growth rate in liquid atrazine minimal media after 24 hr (FIG. 1A and FIG. 1B). Both bacteria developed obvious zones of clearing after 168 hr on LB agar medium amended with atrazine (500 mg/L) (FIG. 1C and FIG. 1D).

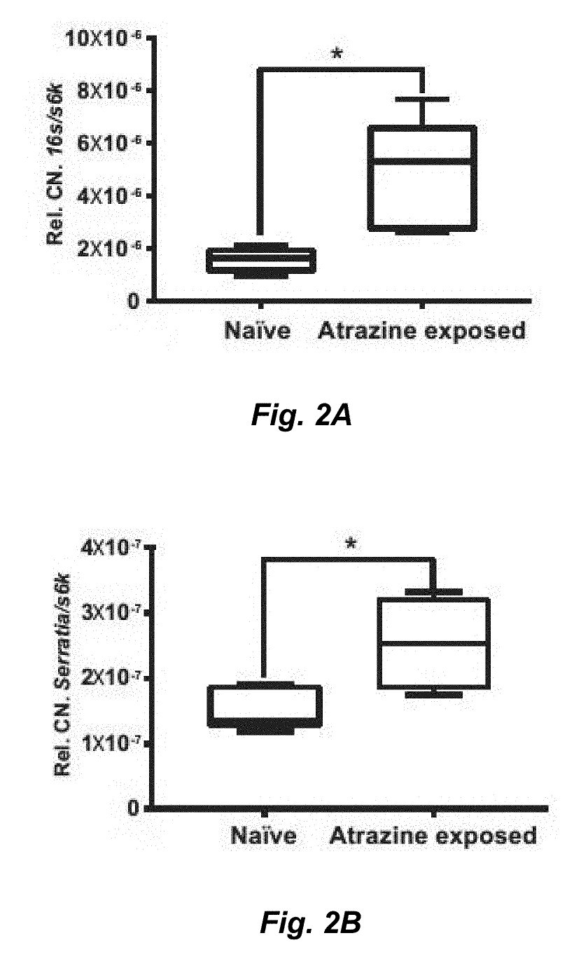

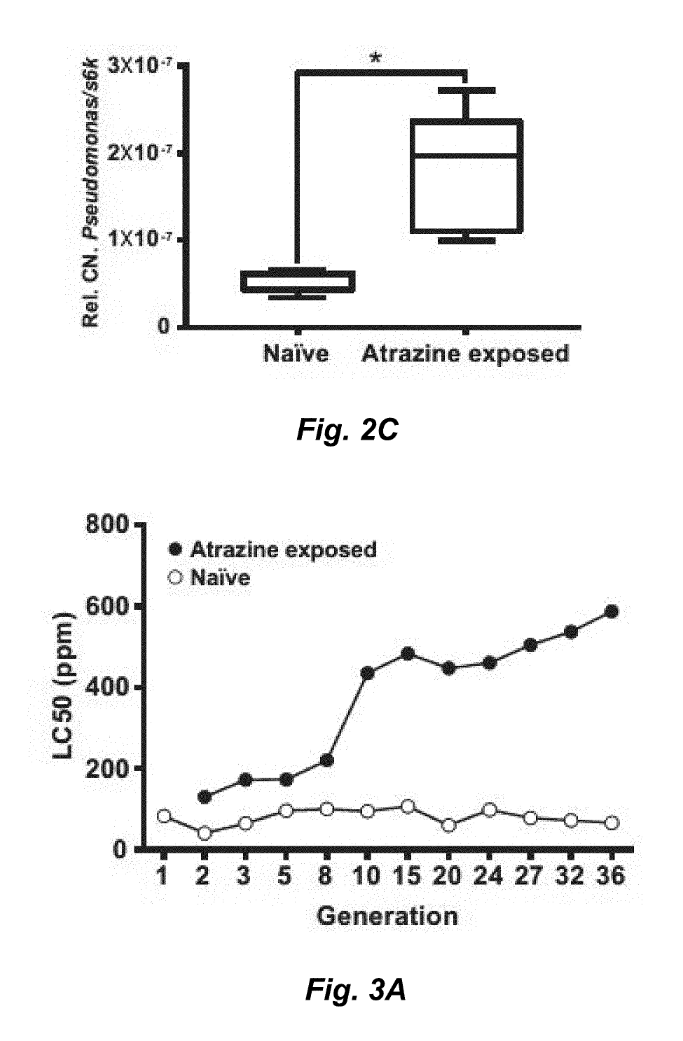

The strain of Serratia increased in abundance in the guts of laboratory animals exposed to the herbicide atrazine after 30 generations while the abundance of Pseudomonas stayed constant (FIG. 2A-FIG. 2C). Furthermore, populations of honeybees and ants collected in corn fields pre and post field application of atrazine also have an increase in Serratia but not Pseudomonas (data not shown). The ubiquitous presence of these genera in animal systems supports their development as a live therapeutic with minimal risk. The relatively low abundance of these two bacteria while still observing the beneficial effect indicates that low dose applications of the probiotics is sufficient for detoxification in at risk organisms.

Development of Probiotic Detoxicant

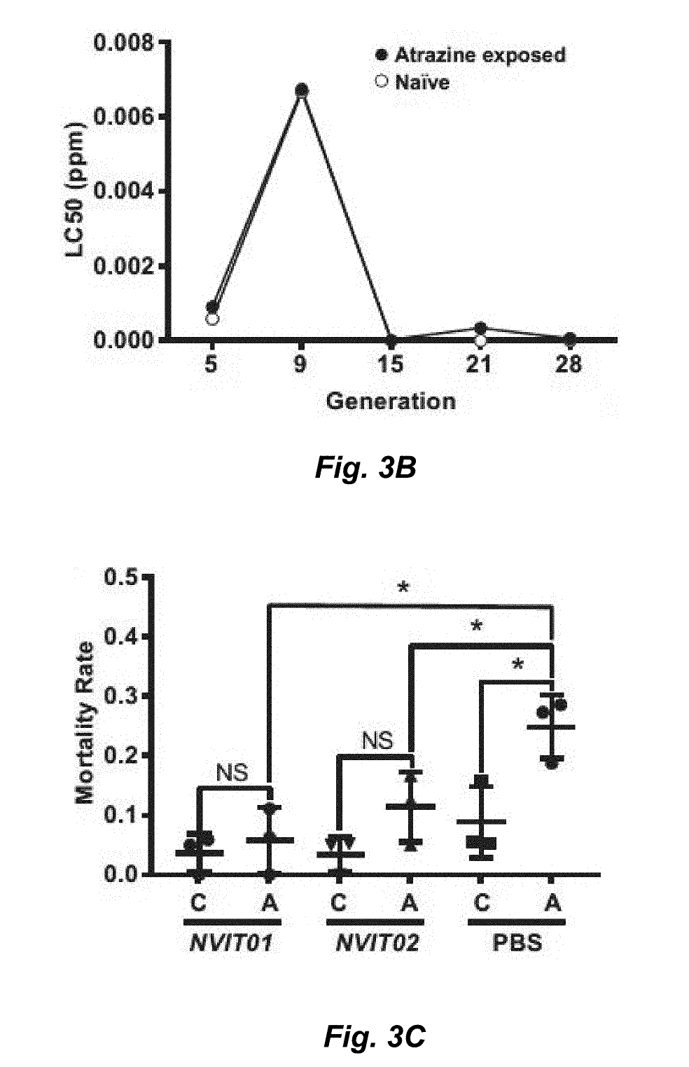

Toxicity experiments were conducted at a range of concentrations for both atrazine and Glyphosate to test the effect that the new microbiome has on increasing resistance. 50 adults (24 hr old) were put into vials from either the generationally-atrazine exposed populations or the naïve populations and exposed to atrazine or glyphosate at parts per million concentrations of 0 ppm, 10 ppm, 30 ppm, 100 ppm, 200 ppm, 400 ppm, 600 ppm, 800 ppm, 1000 ppm. Mortality was recorded after 0, 24, 48, 72, 96, 120, 168 hours (from atrazine exposure point). These were conducted in concurrent replicate experiments. The populations that were previously exposed to atrazine and containing higher levels of Serratia and Pseudomonas had a significantly greater resistance to the herbicides' toxicity (FIG. 3A). The median lethal concentration of atrazine to kill half of the population (LC50) was nearly ten times greater in the generationally-atrazine exposed populations (562 ppm) compared to the naïve population (62 ppm), a significant increase in resistance to the herbicide (p<0.05, resistance ratio probit analysis). Likewise, the generationally-atrazine exposed populations were twice as resistant to Glyphosate, with an LC50 of 315 ppm in the generationally-atrazine exposed populations and 148 ppm in the naïve populations.

To test the role of the microbiome as a detoxicant to herbicides, both populations were tested in germfree, gnotobiotic, conditions. Survival was measured when exposed atrazine concentrations of 0, 5 ppb, 10 ppb, 15 ppb, 25 ppb, 30 ppb for up to 96 hr. Both the generationally-atrazine exposed populations and the naïve populations required their microbiome for any resistance to the herbicide, as animals with their bacteria removed increased toxicity of atrazine over to 100 fold greater than normal (FIG. 3B).

Development of Probiotic Detoxicant

Using the isolates Serratia marcescens NVIT01 and Pseudomonas protegens NVIT02 two probiotic dosing regimens were developed to prevent the toxic effects of herbicide exposure. Each isolate was cultured in a minimal media with 30 ppb atrazine for 72 hr at 25° C. in a shaking incubator. Cells were then pelleted and the media removed. Cells were suspended in sterile saline and repelleted a total of three times to remove all original media growth components and residual atrazine. A final suspension of bacteria was diluted to (P. protegens NVIT02, or S. marcescens NVIT01 (OD620=0.0046, ≈8×10⁶ cells/ml, OD620=0.084, ≈3×10⁸ cells/ml, respectively). 24 hrs old adults were sprayed with PBS, P. protegens NVIT02, or S. marcescens NVIT01. This final cell suspension was then applied to naïve animals using an aerosolizing mister that deposited approximately 800 μl on the animal and its rearing chamber. Animals were observed to noticeably clean the droplets containing the probiotic from their cuticle.

After the animals had been inoculated with the probiotic for 6 hr they were then exposed to atrazine at 100 ppm or a control sugar water and population mortality was observed for 72 hr to calculate the Measurement of Median LC50 (FIG. 3C). Resistance to the herbicide was conveyed through the application of either Serratia marcescens NVIT01 and Pseudomonas protegens NVIT02 as a probiotic. The resistance to herbicides was observed to be due to the detoxifying bacteria in the gut community of both populations.

It is demonstrated herein that bacterial probiotics can be used as a detoxicant for e.g., at risk subjects, including rural water and municipal water treatment, farmers and field hands, and honeybees. It was also observed that changes in the microbiome were heritable across multiple generations, even after ceasing exposure, suggesting that microbiome shifts are inherited transgenerationally. Therefore, subjects (e.g., animals) will benefit from microbiome therapies that are at risk to persistent pesticide exposure throughout a season. Human exposure risk is common, however, peak exposure is at the beginning of a growing season. Probiotic therapies as a preventative to the indirect toxic effects of agro-chemicals can be administered to individuals to assist in detoxifying the compounds. This can also be applied to livestock, and domestic honeybees. Serial or single dose inoculations that establish in the gut microbiome can provide lasting benefit of reduced toxicity risk.

### REFERENCES

Potts, S. G. et al. Global pollinator declines: Trends, impacts and drivers. Trends Ecol. Evol. 25, 345-353 (2010).
Hallmann, C. A. et al. More than 75 percent decline over 27 years in total flying insect biomass in protected areas. PLoS One 12, (2017).
Mac Loughlin, C., Canosa, I. S., Silveyra, G. R., López Greco, L. S. & Rodriguez, E. M. Effects of atrazine on growth and sex differentiation, in juveniles of the freshwater crayfish Cherax quadricarinatus. Ecotoxicol. Environ. Saf. 131, 96-103 (2016).
Mannix, M. A. Draft Ecological Risk Assessments for Atrazine, Simazine, and Propazine Registration Review. EPA-HQ-OPP-2013-0266 (2016) found on the world wide web at www.regulations.gov/docket?D=EPA-HQ-OPP-2013-0266.
Govantes, F., Porrúa, O., Garcia-González, V. & Santero, E. Atrazine biodegradation in the lab and in the field: Enzymatic activities and gene regulation. Microb. Biotechnol. 2, 178-185 (2009).
Vogel, A., Jocque, H., Sirot, L. K. & Fiumera, A. C. Effects of atrazine exposure on male reproductive performance in Drosophila melanogaster. J. Insect Physiol. 72, 14-21 (2015).
Krupke, C. H., Hunt, G. J., Eitzer, B. D., Andino, G. & Given, K. Multiple routes of pesticide exposure for honey bees living near agricultural fields. PLoS One 7, (2012).
Engel, P. & Moran, N. A. The gut microbiota of insects—diversity in structure and function. FEMS Microbiol. Rev. 37, 699-735 (2013).

The present invention can be defined by any of the following numbered paragraphs:
1. A composition for detoxifying an herbicide, the composition comprising viable Serratia marcescens (S. marcescens) bacteria consisting of and a carrier
2. A composition for detoxifying an herbicide, the composition comprising viable Pseudomonas protegens (P. protegens) bacteria and a carrier.
3. A composition for detoxifying an herbicide, the composition comprising a mixture of viable bacteria consisting of S. marcescens, P. protegens, and a carrier.
4. The composition of paragraph 1 or 3, wherein the S. marcescens strain is NVIT01.
5. The composition of paragraph 1 or 3, wherein the S. marcescens is present in the composition at an abundance of at least 3×10⁸ cells/mL.
6. The composition of paragraph 2, wherein the P. protegens strain is NVIT02.
7. The composition of paragraph 2 or 3, wherein the P. protegens is present in the composition at an abundance of at least 8×10⁶ cells/mL.
8. The composition of paragraph 1-3, wherein the carrier is phosphate-buffered saline.
9. The composition of paragraph 1-3, wherein the carrier promotes bacterial growth.
10. The composition of paragraph 1-9, wherein the composition does not elicit an immune response.
11. The composition of any one of paragraphs 1-10, formulated for aerosol delivery.
12. The composition of any one of paragraphs 1-10, formulated for oral delivery.
13. A method of detoxifying an herbicide consumed by a subject, the method comprising administering a composition of any one of paragraphs 1-12 to a subject exposed to an herbicide.
14. The method of paragraph 13, wherein the subject is an insect or a mammal.
15. The method of paragraph 13, wherein the subject is human.
16. The method of paragraph 13, wherein the herbicide is atrazine or Glyphosate.
17. The method of paragraph 13, wherein the administering is an aerosol administration.
18. The method of paragraph 13, wherein the administering is an oral administration.
19. The method of paragraph 13, wherein the administering the compositions detoxifies the herbicide.
20. A method of promoting a resistance to an herbicide, the method comprising administering a composition of any one of paragraphs 1-12 to a subject in need thereof
21. A method of treating a body of water, the method comprising administering a composition of any one of paragraphs 1-12 to a body of water.
22. The method of paragraph 21, wherein the body of water is a municipal water supply.
23. The method of paragraph 21, wherein the body of water contains an herbicide.

1) A composition for detoxifying an herbicide, the composition comprising viable bacteria of the Serratia genus and/or viable bacteria of the Pseudomonas genus and a carrier.

2) The composition of claim 1, wherein the viable bacteria of the Serratia genus is Serratia marcescens (S. marcescens).

3) The composition of claim 2, wherein the S. marcescens strain is NVIT01.

4) The composition of claim 2, wherein the S. marcescens is present in the composition at an abundance of at least 3×10⁸ cells/mL.

5) The composition of claim 1, wherein the viable bacteria of the Pseudomonas genus is Pseudomonas protegens (P. protegens).

6) The composition of claim 5, wherein the P. protegens strain is NVIT02.

7) The composition of claim 5, wherein the P. protegens is present in the composition at an abundance of at least 8×10⁶ cells/mL.

8) The composition of claim 1, wherein the carrier is phosphate-buffered saline.

9) The composition of claim 1, wherein the carrier promotes bacterial growth.

10) The composition of claim 1, wherein the composition does not elicit an immune response.

11) The composition of claim 1, formulated for aerosol delivery or for oral delivery.

12) A method of detoxifying an herbicide consumed by a subject, the method comprising administering a composition of claim 1 to a subject exposed to an herbicide.

13) The method of claim 10, wherein the subject is an insect, a mammal or a human.

14) The method of claim 10, wherein the herbicide is atrazine or Glyphosate.

15) The method of claim 10, wherein the administering is an aerosol administration or an oral administration.

16) The method of claim 10, wherein the administering the compositions detoxifies the herbicide.

17) A method of promoting a resistance to an herbicide, the method comprising administering a composition of claim 1 to a subject in need thereof.

18) A method of treating a body of water, the method comprising administering a composition of claim 1 to a body of water.

19) The method of claim 18, wherein the body of water is a municipal water supply.

20) The method of claim 18, wherein the body of water contains an herbicide.


SEQUENCE LISTING (2 sequences)

SEQ ID NO: 1 - DNA, 1376 bp, Serratia marcescens
tcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacatactttatgaggtccgcttgctctcgcgaggtcgcttctctttgtatatgccattgtagcacgtgtgtagccctactcgtaagggccatgatgacttgacgtcatccccaccttcctccagtttatcactggcagtctcctttgagttcccggccgaaccgctggcaacaaaggataagggttgcgctcgttgcgggacttaacccaacatttcacaacacgagctgacgacagccatgcagcacctgtctcagagttcccgaaggcaccaatccatctctggaaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgatttaacgcgttagctccggaagccacgcctcaagggcacaacctccaaatcgacatcgtttacagcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactctagcttgccagtttcaaatgcagttcccaggttgagcccggggatttcacatctgacttaacaaaccgcctgcgtgcgctttacgcccagtaattccgattaacgcttgcaccctccgtattaccgcggctgctggcacggagttagccggtgcttcttctgcgagtaacgtcaattgatgaacgtattaagttcaccaccttcctcctcgctgaaagtgctttacaacccgaaggccttcttcacacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattaccccacctactagctaatcccatctgggcacatctgatggcaagaggcccgaaggtccccctctttggtcttgcgacgttatgcggtattagctaccgtttccagtagttatccccctccatcaggcagtttcccagacattactcacccgtccgccgctcgtcacccagggagcaagct

SEQ ID NO: 2 - DNA, 1304 bp, Pseudomonas protegens
gcggcggacgggtgagtaatgcctaggaatctgcctagtagtgggggataacgtccggaaacgggcgctaataccgcatacgtcctacgggagaaagtgggggatcttcggacctcacgctattagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgccc